(19) United States
(12) Patent Application Publication
(10) Pub. No.: US 2023/0277705 A1
(43) Pub. Date: Sep. 7, 2023

(54) DISINFECTION DEVICE AND PROTECTIVE SLEEVE COMPRISING A DISINFECTION DEVICE

(71) Applicant: Peter Ammann, Oberhofen (CH)

(72) Inventor: Peter Ammann, Oberhofen (CH)

(21) Appl. No.: 18/011,488

(22) PCT Filed: Jun. 18, 2021
(86) PCT No.: PCT/EP2021/066652

§ 371 Date: Dec. 19, 2022

(30) Foreign Application Priority Data
Jun. 22, 2020 (WO) PCT/EP2020/067322

(51) Int. Cl. A61L 2/22; A61L 2/26; A61M 35/00; H04B 1/3888; B65D 83/38; B05B 15/16; B05B 11/00; B65D 83/20; B05B 1/14

(52) U.S. Cl. CPC A61L 2/22; A61L 2/26; A61M 35/006; B05B 1/14; B05B 11/0059; B05B 11/1023; B05B 15/16; B65D 83/20; B65D 83/388; H04B 1/3888; A61L 2202/15

(57) ABSTRACT

A disinfection device (la) for applying a disinfectant to at least one fingertip (F) of a user comprises a housing (6; 6a, 6b) with a recess (4) accessible from the outside through an opening (3), into which recess the fingertip can be inserted, a spray unit (8) inside the housing, and a container (7) for disinfectant fluidly connected to the spray unit. The spray unit comprises a spray head (2) which projects into the recess and a valve device (9a) which is fluid-connected to the spray head and is designed in such a way that, when the spray head is actuated by means of the fingertip, it releases disinfectant from the container as an aerosol into the recess so that the aerosol can wet the fingertip.

### TECHNICAL FIELD

The present invention relates to a disinfection device and to a protective cover for a communication device with such a disinfection device according to the respective independent claim.

### BACKGROUND

Disinfection devices are well known. Most of them are conventional spray cans filled with a disinfectant that is released when the spray head is pressed. Furthermore, dispensers are also known which are stationary in hospitals, etc. and contain a relatively large amount of disinfectant so that they last for a longer period of time during intensive use in frequented environments. These solutions have the disadvantage that they are not suitable as portable solutions due to their size. In addition, dosing can be difficult and in many cases, an excessive amount of agent is dispensed with one actuation.

### DESCRIPTION OF THE INVENTION

The objective is therefore to provide an improved disinfection device. In particular, the objective is to provide a disinfection device that is easy to handle, effective, selectively applicable and reusable.

This objective is reached in a first aspect of the invention with a disinfection device according to claim 1. Accordingly, the disinfection device for applying a disinfectant to at least one fingertip of a user comprises a housing with a recess accessible through an opening from the outside, into which recess the fingertip is inserted, inside the housing a spray unit and a container for disinfectant fluidly connected to the spray unit. The spray unit comprises a spray head which projects into the recess and a valve device fluidly connected to the spray head. The valve device is configured to release disinfectant from the container as an aerosol into the recess when the spray head is actuated by the fingertip, so that the aerosol can wet the fingertip. The housing includes a first section for holding the container and a second section for holding the spray unit, the first and second sections being separably attachable to each other by means of cooperating connecting elements so that the container is replaceable by disengaging the connection of the two sections.

In a preferred embodiment, the recess is filled with a sponge element in which the spray head is completely embedded.

In a preferred embodiment, the second section forming the recess is not movable and the spray head is movable relative to the second section when actuated to release disinfectant.

In a second aspect of the invention, the objective is solved with a protective cover for a mobile communication device, in particular for a cell phone or a tablet. The protective cover comprises, in a region facing a rear side of the communication device, an integrated disinfection device for applying a disinfectant to at least one fingertip of a user. The disinfection device comprises a container for a disinfectant integrated in the protective cover, a spray unit fluidly connected to the container, wherein the spray unit comprises a spray head and a valve device fluidly connected to the spray head. The protective cover has a recess accessible through an opening from the outside, into which the spray head projects and in which a fingertip of the user is received. The valve device is configured such that when the spray head is actuated by means of the fingertip, it releases disinfectant from the container as an aerosol into the recess so that the aerosol can wet the fingertip.

The present invention in both aspects has the advantage that a user can use the disinfection device, if necessary, in any situation where in daily life there is a risk of contamination and possible infection with pathogens due to the frequent unavoidable touching of objects, and this can be done quickly and selectively by being easily usable for the disinfection of a single finger and by avoiding undesirable disinfection over a large area. Furthermore, the configuration of the disinfection device as a two-part device ensures that the container with the disinfectant can be easily replaced.

Both aspects of the invention cover disinfection on the user himself, whereby the user can, for example, before and/or after pressing a button, e.g. when operating an ATM or buttons in elevators where there is a high risk of contamination with pathogens because they are used by many people, disinfect the finger that came into contact with the button. Thus, when a button of such a device is pressed, the user can subsequently use the disinfection device to disinfect the fingertip that was used to press the button. To do this, all he has to do is briefly press the spray head with the fingertip in question so that disinfectant can wet the fingertip and thus disinfect it. Apart from its obvious ease of use, the disinfection device has the further important advantage that it only needs to be used selectively for the fingertip concerned. As described above, conventional devices often dispense too much disinfectant, which has to be collected in the hand and can also drip onto the floor, wasting disinfectant. Further it is a disadvantage that with the liquid agent in the hand the whole hand or both hands must be disinfected by rubbing. These disadvantages are avoided by the present disinfection device, which saves a large amount of disinfectant, since just for the described scenario actually only a small section of a hand (the fingertip) comes into contact with the potentially contaminated button and has to be disinfected. Last but not least, it is also of great advantage that especially with frequent disinfection, the drying out of the hands, which is known to occur due to frequent disinfection of the hands with typically alcohol-containing agents, is largely avoided.

The second aspect of the invention has the additional advantage of providing a portable disinfecting solution that is already integrated into an item that is used daily. Since in today’s world a cell phone is a constant companion, integrating the disinfection device into the protective case of the cell phone results in an effective disinfection solution without the need to carry an additional device.

### BRIEF DESCRIPTION OF THE DRAWINGS

Further embodiments, advantages and applications of the invention are apparent from the dependent claims and from the description which now follows with reference to the figures. Thereby it is shown in:

FIG. 1a a sectional view of a disinfection device according to the first aspect of the invention immediately before disinfection,

FIG. 1b a sectional view of the disinfection device of FIG. 1a according to the invention during disinfection,

FIG. 2 a detailed sectional view of the disinfection device of FIG. 1a,

FIGS. 3a,b a side view and a perspective view, respectively, of a further embodiment of the disinfection device according to the first aspect of the invention,

FIGS. 4a,b a rear side view and a side view, respectively, of a protective cover according to the second aspect of the invention with a disinfection device,

FIG. 5 an application example of the disinfection device of FIG. 1,

FIG. 6 a further embodiment of the disinfection device of FIG. 1,

FIG. 7 a further embodiment of the disinfection device of FIG. 1, and

FIG. 8 an example of use of the disinfection device of FIG. 7.

### WAYS TO CARRY OUT THE INVENTION

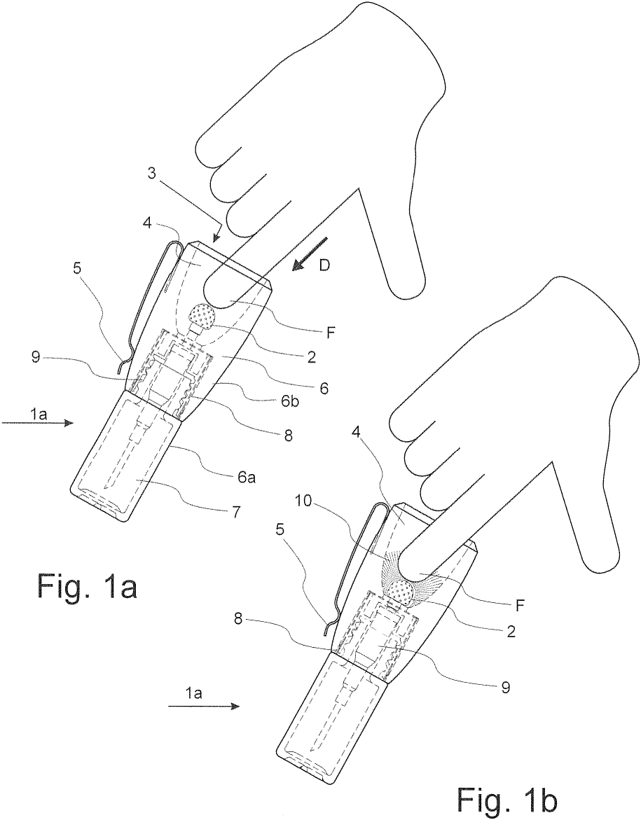

FIGS. 1a and 1b are explained together below. They show a disinfection device 1a with a housing 6, a spray unit 8 and a container 7 for disinfectant, which is fluid-connected to the spray unit.

In all embodiments and aspects of the invention, the housing 6 comprises a first section 6a holding the container 7 and a second section 6b holding the spray unit 8, the first and second sections being separably attachable to each other by means of cooperating connection elements 9. The container becomes accessible and can be replaced by disengaging the connection of the two sections. It is preferred if the cooperating connection elements form a screw closure. However, they can also form a snap-in connection. However, it is also possible that the first section 6a of the housing is itself the container, which has connecting means (e.g. threads) for connection to the second section 6b of the housing, which results in a material saving for the housing material.

To actuate the spray head for dispensing disinfectant, the spray head can be pressed by means of the fingertip in the direction of the valve device from a basic position, in which the valve device is closed, to a spray position, in which the valve device is open and releases disinfectant from the container. In this case, the spray head automatically returns to the basic position without external actuation.

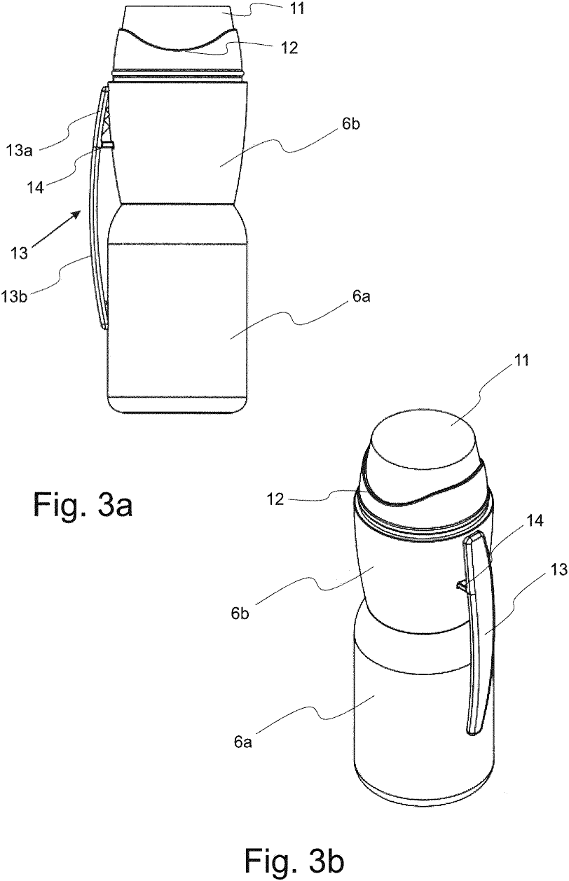

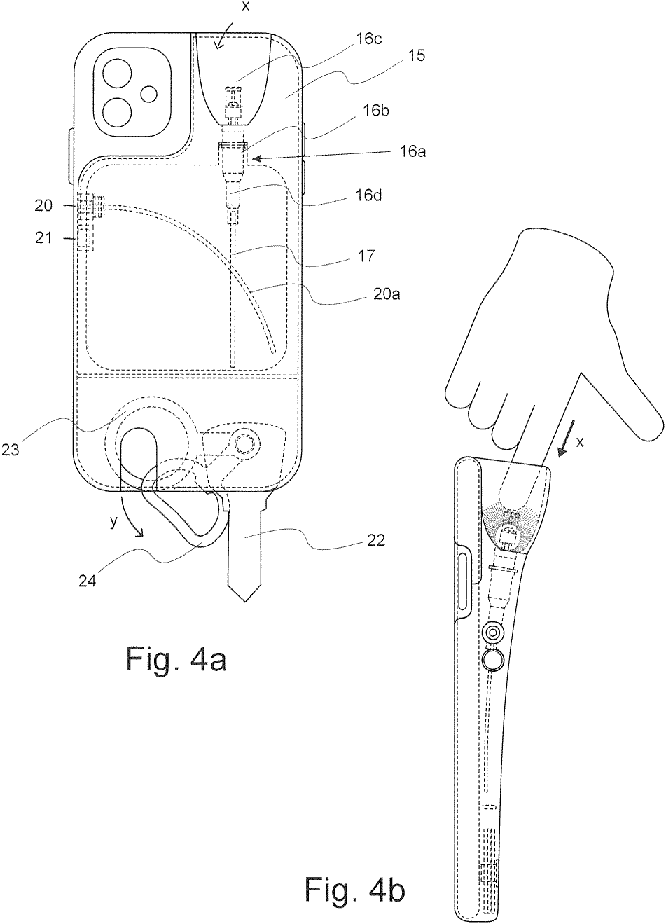

The container for disinfectant is preferably refillable. It is fluid-connected to the spray unit 8. Fluid-connected throughout the present context means that fluid can enter the spray unit from the container. The container may be a pressurized container and may be used with blowing agent, or it may be a conventional container in the manner of perfume bottles in which the spraying effect is obtained by pumping air into the interior of the container. For this purpose, the spraying unit may be suitably configured to release disinfectant continuously during a single actuation for the duration of the actuation or to release only one dose of disinfectant. The advantage of the first variant is explained in connection with a preferred feature of the second section 6b of the housing in the context of FIGS. 3a, 3b. The second variant is advantageous because a short spraying operation is sufficient here for one or more fingertips in each case. It is further preferred if the spray unit can release disinfectant upwardly or downwardly or laterally, which also applies to the disinfection device according to the first aspect of the invention but is explained in detail in connection with the second aspect of the invention in the context of FIGS. 4a,b.

As can be seen from the synopsis of FIGS. 1a and 1b, the user inserts the fingertip F in the direction of the arrow D through an opening 3 into a recess 4 of the housing 6 or the second section 6b of the housing and presses a spray head 2 of the spray unit 8 in the direction of the container 7, which causes the disinfectant to be released as an aerosol 10. As a result, his fingertip is disinfected. Of course, the disinfection device or the recess can be dimensioned in such a way that several fingertips can be inserted into the recess 4 at the same time. The second section forming the recess is preferably not movable and the spray head is movable relative to the second section when actuated to release disinfectant, which is evident from the synopsis of FIGS. 1a and 1b.

In embodiments, the central axes of the housing 6 and the spray unit 8 and the container 7 may coincide. This simplifies the construction of the disinfection device and is space-saving. However, it is also conceivable to provide the first section 6a of the housing with the container, for example, at a 90° angle to the second section 6b of the housing.

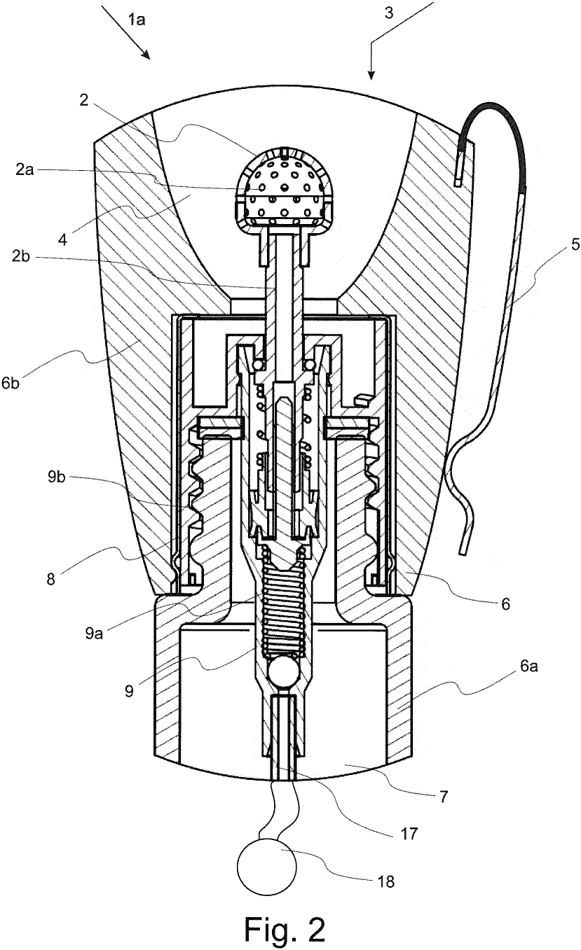

FIG. 2 shows a detailed sectional view of the disinfection device of FIG. 1a with the two housing sections 6a and 6b, which are connected by means of connecting elements 9b designed as a screw connection. As mentioned, here the first section 6a of the housing carries the container and the spray unit. The spray head 2 is preferably spherical or elliptical and fluidly connected to the valve device of the spray unit by means of a tube 2b. The spray head comprises a plurality of nozzles 2a through which disinfectant can be sprayed into the recess 4. This embodiment of the spray head is preferred for all aspects of the disinfection device. It has the advantage that the aerosol is sprayed in many directions, thus ensuring comprehensive disinfection, which would not be possible to the same extent with a conventional unidirectional spray head due to the limited clearance for the fingertip in recess 4. However, it should be mentioned in this context that specifically in this embodiment, the first section 6a of the housing together with the container and spray unit can be unscrewed and provided with a conventional unidirectional spray head instead of the spherical spray head, so that the disinfection device can also be used as a conventional spray can. As mentioned, the spray unit includes a valve device 9a which, when the spray head is actuated in the direction of the container, allows disinfectant to be dispensed from the latter through the tube 2b into the spray head 2 and through the nozzles 2a as an aerosol into the recess.

In particular, these can be embodiments in which the orientation is not important, i.e., spraying can also be carried out upside down with the disinfection device. Preferably, the valve device is fluid-connected to a flexible hose 17, which is arranged inside the container and through which disinfectant reaches the spray head when the spray head is actuated. The hose has a weight element 18 at its free end, which is designed in such a way that, as a result of its weight force, it is immersed in the disinfectant up to that wall of the container which is closest to the ground, irrespective of the orientation of the disinfection device. The weight element is preferably formed by a ball 18, in particular a metal ball, although other shapes are also conceivable. The ball is preferably enclosed with clearance in a ball housing provided with holes (not shown), so that when the spray head is actuated, disinfectant flows from the container through the holes of the ball housing between the ball and the ball housing into the flexible hose. This allows the disinfection device to be used easily in any position and regardless of fill level.

FIGS. 3a and 3b show a side view and a perspective view, respectively, of a disinfection device according to the first aspect of the invention with additional preferred features.

In this embodiment, the recess is filled with a sponge element 11 in which the spray head is completely embedded. It is preferred if the sponge element is replaceable. Due to its soft texture, the sponge element contributes to comfort during disinfection and enables uniform wetting of the skin area to be disinfected.

Furthermore, in embodiments, the second section 6b of the housing may be such that the edge defining the recess is not straight in at least one region 12 but is rounded downwardly toward the first section 6a. Preferably, two opposing such regions 12 are provided. As a result, the disinfection device can also be used for the disinfection of, for example, handles, since the edge of the recess can adapt to the rounded shape of a handle. This is particularly effective in combination with the sponge element 11, since better wetting of the handle can be achieved by means of the sponge element. In this context, an advantage of using a container configured as a pressurized container is also clear, in that disinfectant is continuously released during a single actuation for the duration of the actuation. In this scenario, the objective is to disinfect surfaces where the disinfection device is guided on the one to be disinfected, so that multiple pumping operations during simultaneous guidance would be more cumbersome. This scenario covers a priori disinfection of an object before it is touched. Thus, the disinfection device can be used for a priori disinfection and also for a posteriori disinfection where a fingertip is disinfected only after an object is touched.

In embodiments, the container comprises on its outside an elastic clip 13, in particular designed as an outwardly curved leaf spring, for clamping a cleaning cloth, which is fastened to the housing by means of a fastening element 14. The fastening element divides the clamp, as seen in its longitudinal extension, into an actuating section 13b with a first free end and a holding section 13a with a second free end. The cleaning cloth (not shown) can be clamped between the second free end and the container wall in a rest position of the clamp. The actuating section is longer than the holding section, such that by pressing the clamp in the actuating section towards the housing, the second end moves away from the housing due to the elasticity of the fastening element, so that the cleaning cloth can be released without being touched by the user and can be disposed in this manner. It is preferred when the holding section faces the recess, while the actuating section faces away from the recess. The reason for this is that the cleaning cloth can be clamped in such a way that it can be placed without contact over the recess or over the sponge element by suitable rotation of the disinfection device. Subsequently, it can also be used to effectively disinfect a flat surface and finally be dropped into a trash can by pressing the actuating section. This has the advantage that the cleaning cloth need not be touched at any time during disinfection or thereafter. Furthermore, the clip 13 can also be used as a holding element for the disinfection device and can be clipped to a belt, for example. It is noted that FIG. 2 shows an alternative clamp which can also be used, but which is not suitable for disinfection by means of a cleaning cloth.

FIGS. 4a and 4b show a rear side view and a side view, respectively, of a protective cover 15 according to the second aspect of the invention with a disinfection device. The dashed lines in the figures indicate that the element in question is located inside the protective cover. As mentioned at the beginning, the protective cover is provided for a mobile communication device, in particular for a cell phone or a tablet. It comprises, in a region facing a rear side of the communication device, an integrated disinfection device for applying a disinfectant to at least one fingertip of a user. The disinfection device comprises a container 16a for a disinfectant integrated in the protective cover, and a spray unit 16b fluidly connected to said container. The spray unit includes a spray head 16c and a valve device 16d fluidly connected to the spray head. The protective cover has a recess accessible from the outside through an opening into which the spray head projects and in which a fingertip of the user can be received, which is indicated by the arrow x. The valve device is designed in such a way that when the spray head is actuated by means of the fingertip, it releases disinfectant from the container as an aerosol into the recess so that the aerosol can wet the fingertip, which is illustrated in FIG. 4b.

In a preferred embodiment, the container of the disinfection device of the protective cover further comprises a refill valve 20 fluidly connected to the container for refilling disinfectant into the integrated container, the refill valve being connected to a flexible hose 20a that extends into the integrated container. The refill valve is designed in such a way that it can be opened by a suitable attachment of a refill container for filling the integrated container and closes again automatically as soon as the refill container is removed. Advantageously, the disinfection device of the protective cover can be refilled in this way.

Alternatively or additionally, the container of the disinfection device of the protective cover comprises a refill closure 21, which releases a filling opening of the container without a valve for refilling disinfectant into the container. Advantageously, this variant enables the container to be refilled from any refill container without a special valve-opening attachment.

Preferably, the container of the disinfection device of the protective cover further comprises a vent valve that connects the integrated container to the environment for venting (not shown).

In this aspect of the invention, the recess is also preferably filled with a sponge element (not shown) in which the spray head is completely embedded. The sponge element is also preferably replaceable.

The protective cover further preferably comprises a cover cap (not shown) for the recess, so that the spray head is protected and is not accidentally actuated. The cover cap may also be provided for the disinfection device according to the first aspect of the invention.

In embodiments, the protective cover is double-walled at least in the area of the integrated container to accommodate the integrated container. This is advantageous as additional security against leakage of the disinfectant. Preferably, the integrated container may be replaceable in this embodiment. Alternatively, the protective cover is double-walled at least in areas and a corresponding intermediate space itself forms the integrated container. Advantageously, no additional container is necessary with this alternative, whereby the protective cover in this case is of course made of a material that is impermeable to the disinfectant.

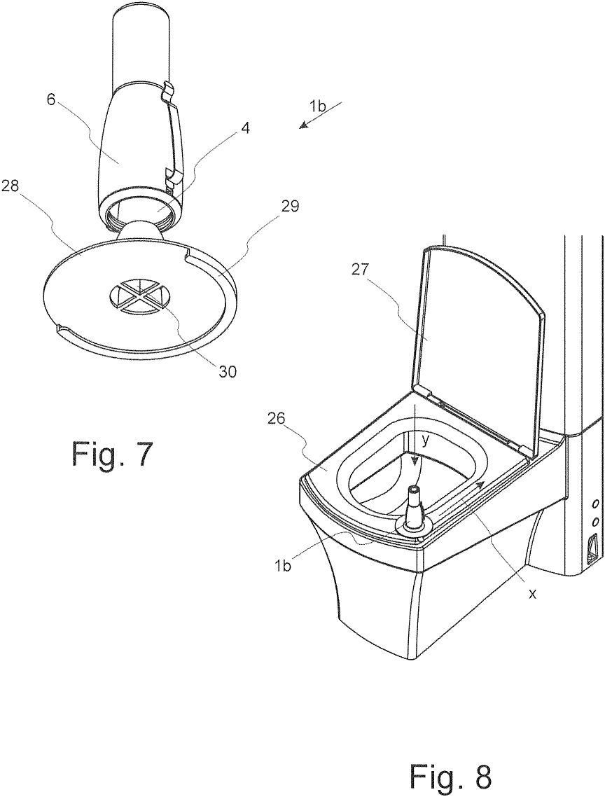

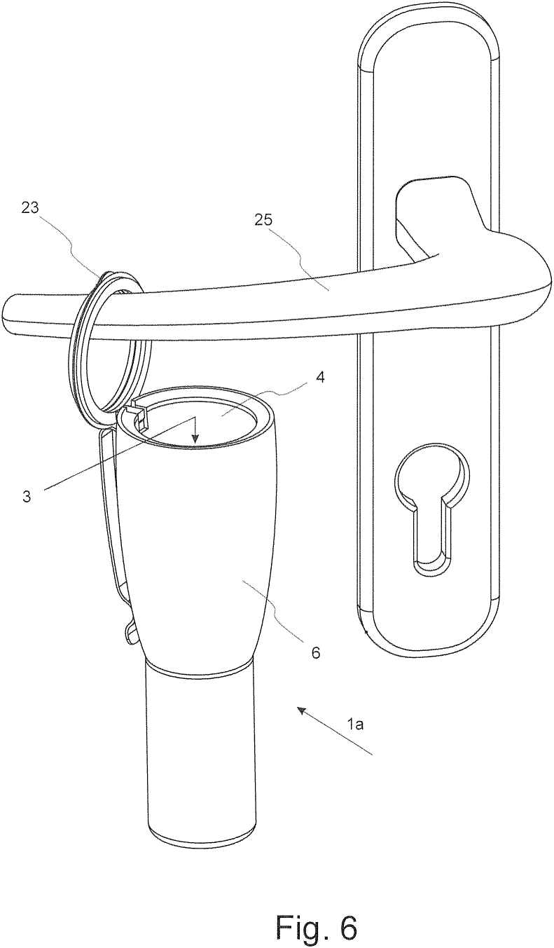

Further, the protective cover preferably comprises a chamber for storing keys 22 or beverage openers 23 or the like, which can be folded out for use (indicated by the arrow y). It may also include a ring 23, which will be discussed in more detail in connection with FIG. 6.

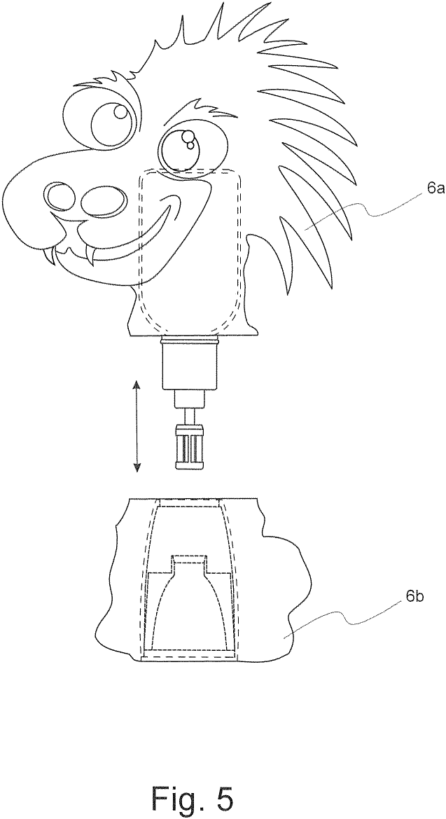

FIG. 5 shows an example of use of the disinfection device according to the first aspect of the invention. This is a mascot for children and can serve as an educational measure to introduce the concept of disinfection to children as well. In this application, the housing is formed by the mascot. The first section 6a holding the container is formed by the head of the mascot. The second section 6b for holding the spray unit is formed by the body of the mascot. The first and second sections are separably attachable to each other by means of cooperating fasteners 9, so that by disengaging the connection of the two sections, the container is interchangeable.

FIG. 6 shows a further embodiment of the disinfection device according to the first aspect of the invention. For the protective cover according to the second aspect of the invention, this concept has already been indicated in connection with FIG. 4a, wherein the use of the ring of the protective cover is the same as described below. Here, a ring 23 is attached to the housing 6, the ring having a diameter such that it can be pulled over a handle 25, in particular a door handle, to operate the handle without direct contact by the user. Further, the ring is configured so as not to leave scratches on the actuated handle. For this purpose, it may be coated with a rubber layer, for example. The use of such a ring is advantageous for the scenario of disinfecting door handles, since these can have very different shapes and can, for example, be comparatively strongly curved and have strongly varying diameters. Therefore, the diameter of the ring is chosen very generously, as can be seen in the figure, so that as much as possible any type of handle can be grasped by the ring. In addition, this makes it very easy to pull the ring over the handle.

FIG. 7 shows a disinfection device 1b in an embodiment in which an adapter element is provided that can be attached to the housing of the disinfection device, and FIG. 8 shows an example of use of the disinfection device from FIG. 7. The scenario already described in the context of the explanation of the downwardly rounded edge of the recess from FIGS. 3a,b dealt with round surfaces to be disinfected, e.g. handles in a public transport vehicle. In this embodiment, flat surfaces are considered, e.g., when disinfecting a toilet seat surface 26 of a toilet 27. The following explanations are, of course, applicable to other flat surfaces such as tables or chairs.

The adapter element comprises a rigid extension element 30 projecting into the recess for actuating the spray head and at least one passage opening towards the object. When the spray head is actuated by means of the extension element by pressing the housing in the direction of the surface to be disinfected, disinfectant passes from the recess through the passage opening onto the surface.

As an alternative to the extension element, the disinfection device comprises an alternative spray head which can be plugged onto the spray head or connected to the valve device instead of the spray head. The alternative spray head protrudes from the opening when plugged on or connected to the valve device, so that it can be used by pressing it outside the recess. This embodiment is generally applicable to all embodiments of the disinfection device, including the protective cover.

The adapter element of FIG. 7 further comprises a plate-shaped support element 28, by means of which the disinfection device can be moved back and forth on a flat surface of the object to be disinfected (arrow x in FIG. 8) in order to disinfect the surface. The support element 28 may also be curved for use on a surface that is also curved. The adapter element has, on a surface facing the object, a layer 29 of a foamed or spongy or rubber-based material or nonwoven material for distributing and/or absorbing disinfectant from the surface of the object, preferably wherein the layer is replaceable with a new layer. This measure further improves the most comprehensive distribution of the disinfectant on the surface to be disinfected. Furthermore, such a layer protects the surface from scratches that could occur due to the back and forth movement of the disinfection device. In this embodiment of the adapter element 28, the layer 29, for example a rubber layer, is designed as a circular sector. In this particular embodiment of the layer, it is advantageous that if the direction of displacement of the disinfection device on the surface is suitable, i.e. in a direction in which there is no layer, the layer can trap the disinfectant already on the surface which has previously been sprayed onto it through the passage openings, so that no wet or damp seating surface remains behind the disinfection device. In addition, the layer in this example also acts as a spacer to the seating surface 26. In this embodiment, the adapter element is preferably designed as a disposable element, since this scenario in particular involves a very germ-laden surface. The use as a one-way element is nevertheless cost-effective with regard to the simplicity of the design of the adapter element as a simple disc, since this adapter can be moulded, for example, from plastic in one step and also does not have to meet any special requirements on the material side with regard to elasticity, etc. Alternatively or additionally, only the rubber layer can serve as a one-way element and may be replaced. In this context, it is noted that the clamp described in the context of FIGS. 3a,b in conjunction with the cleaning cloth is also regarded as an alternative to the embodiment according to FIGS. 7 and 8, with the advantage that no support element is necessary in this case.

The present invention has a number of advantages over already known solutions. It offers great flexibility of use in a wide variety of disinfection scenarios, depending on what is most appropriate for a particular situation, which also saves time for the user; disinfection on the user or on an object to be touched by the user. Fingertip disinfection is an a posteriori disinfection that takes place after touching, while disinfection of objects to be touched is mainly an a priori disinfection that takes place before touching. Furthermore, the invention can save disinfectant and the disinfection on the user itself can be carried out more gently for the hands, in that only the fingertips concerned, which have actually had contact with a foreign object, are disinfected selectively, so that the skin of the rest of the hand is not affected.

While preferred embodiments of the invention are described in the present application, it should be clearly noted that the invention is not limited to these and may be carried out in other ways as well within the scope of the following claims.

1. Disinfection device (1a) for applying a disinfectant to at least one fingertip (F) of a user, comprising
a housing (6; 6a, 6b) with a recess (4) accessible from the outside through an opening (3), into which recess the fingertip is inserted,
a spray unit (8) inside the housing,
a container (7) for disinfectant fluidly connected to the spray unit,
wherein the spray unit comprises a spray head (2) projecting into the recess and a valve device (9a) fluidly connected to the spray head, which valve device is configured to release disinfectant from the container as an aerosol into the recess when the spray head is actuated by the fingertip, so that the aerosol can wet the fingertip,
wherein said housing comprises a first section (6a) holding said container and a second section (6b) for holding said spraying unit, wherein said first and second sections are separably attachable to each other by means of cooperating connecting elements (9) so that by disengaging the connection of said two sections said container is replaceable.

2. Disinfection device according to claim 1, wherein the recess is filled with a sponge element in which the spray head is completely embedded, in particular wherein the sponge element is replaceable.

3. Disinfection device according to claim 1, wherein the valve device is fluid-connected to a flexible hose which is arranged inside the container and through which disinfectant reaches the spray head when the spray head is actuated, and wherein the hose has at its free end a weight element which is configured in such a way that, as a result of its weight force, it is immersed in the disinfectant up to a wall of the container which is closest to the ground, irrespective of the orientation of the disinfection device.

4. Disinfection device according to claim 3, wherein the weight element is formed by a metal ball, wherein the ball is enclosed with clearance in a ball housing provided with holes, so that when the spray head is actuated, disinfectant flows from the container through the holes of the ball housing between the ball and the ball housing into the flexible hose.

5. Disinfection device according to claim 1, wherein the container comprises on its outside an elastic clip, in the shape of an outwardly curved leaf spring, for clamping a cleaning cloth, which clip is fastened to the housing by a fastening element, wherein the fastening element divides the clip, as seen in its longitudinal extension, into an actuating section with a first free end and a holding section with a second free end, wherein the cleaning cloth is clampable between the second free end and the container wall in a rest position of the clamp, wherein the actuating section is longer than the holding section, such that by pressing the clamp in the actuating section towards the housing, the second end moves away from the housing so that the cleaning cloth is releasable without contact by the user.

6. Disinfection device according to claim 1, wherein a ring (23) is attached to the housing, wherein the ring has a diameter such that it can be pulled over a handle (22), or a door handle, to operate the handle without direct contact by the user.

7. Disinfection device according to claim 1, further comprising an alternative spray head attachable to the spray head or connectable to the valve device in place of the spray head, wherein the alternative spray head protrudes from the opening when plugged on or connected to the valve device so that it is usable by pressing outside the recess.

8. Disinfection device according to claim 1, wherein asecond section (6b) forming the recess is not movable and the spray head is movable relative to the second section when actuated to release disinfectant.

9. Protective cover for a mobile communication device, a cell phone or a tablet, wherein the protective cover comprises, in a region facing a rear side of the communication device, an integrated disinfection device for applying a disinfectant to at least one fingertip of a user, the disinfection device comprising:
a container for a disinfectant integrated in the protective cover,
a spray unit fluidly connected to the container, wherein the spray unit comprises a spray head and a valve device fluidly connected to the spray head,
wherein the protective cover has a recess which is accessible from the outside through an opening and into which the spray head projects and in which a fingertip of the user is received,
wherein the valve device is configured such that it releases disinfectant from the container as an aerosol into the recess when the spray head is actuated by means of the fingertip, so that the aerosol can wet the fingertip.

10. Protective cover according to claim 9, wherein the container of the disinfection device comprises a refill valve fluidly connected to the integrated container for refilling disinfectant into the container, wherein the refill valve is connected to a flexible tube extending into the integrated container.

11. Protective cover according to claim 10, wherein the container of the disinfection device comprises a refill closure which valve-free opens a filling opening of the container for refilling disinfectant into the container.

12. Protective cover according to claim 9, wherein the container of the disinfection device comprises a vent valve connecting the integrated container to the environment for venting.

13. Protective cover according to claim 9, wherein the recess is filled with a sponge element in which the spray head is completely embedded, such that the sponge element is replaceable.

14. Protective cover according to claim 9, further comprising a cover cap for the recess.

15. Protective cover according to claim 9, wherein the protective cover is double-walled at least in the region of the integrated container for receiving the integrated container, or wherein the protective cover is double-walled at least in regions and a corresponding intermediate space itself forms the integrated container.
